(19) United States
(12) Patent Application Publication
(10) Pub. No.: US 2019/0030165 A1
(43) Pub. Date: Jan. 31, 2019

(54) NEGATIVE CONTROL CANNABIS FORMULATIONS

(71) Applicant: Vireo Health LLC, Minneapolis, MN (US)

(72) Inventors: Kyle Kingsley, Minneapolis, MN (US); Eric Greenbaum, New York, NY (US)

(73) Assignee: Vireo Health LLC, Minneapolis, MN (US)

(21) Appl. No.: 15/592,133

(22) Filed: May 10, 2017

Related U.S. Application Data
(60) Provisional application No. 62/333,923, filed on May 10, 2016.

(51) Int. Cl. A61K 47/06; A61K 47/28; A61K 47/22; A61K 49/00; A61K 36/185; A23L 33/105; A23L 27/20

(52) U.S. Cl. CPC A61K 47/06; A61K 47/28; A61K 47/22; A23V 2002/00; A61K 36/185; A23L 33/105; A23L 27/20; A61K 49/00

(57) ABSTRACT

Compositions and methods useful for providing negative control formulations for clinical trials involving whole plant cannabis extracts are provided. The formulations include a plurality of odorants and flavorants designed to mimic the unique odor and flavor profile of whole plant cannabis extracts. The odorants and flavorants may include the terpenes and terpinoids.

### STATEMENT REGARDING FEDERALLY SPONSORED RESEARCH

N/A

### FIELD OF THE INVENTION

The present invention relates generally to formulations that mimic the cannabis odorant and/or flavorant profiles to be used as negative controls in experiments featuring cannabis based test substances.

### BACKGROUND OF THE INVENTION

In the past few decades the medicinal value of cannabis has gained broader acceptance in the medical and scientific communities as evidence of its medical utility has accumulated. While various cannabinoids have displayed intriguing biological activities, either alone or in various combinations, whole plant extracts (WPE) of cannabis strains may prove to be particularly useful from both a scientific and commercial perspective. As WPE are further developed they will need to undergo rigorous placebo controlled trials to prove their safety and efficacy. An issue with using whole plant extracts, particularly when formulated for vaporization, nebulization, and/or oral administration is the particular flavor and odor profile associated with the extracts. This flavor/odor profile has the tendency to inform subjects that they are receiving the test substance as opposed to the placebo and can bias the trial.

### SUMMARY OF THE INVENTION

As specified in the Background Section above, there is a need in the art for a product that has the odor and flavor profiles of cannabis but does not contain the bioactive cannabinoids.

Thus According to a first embodiment, the present invention provides a composition comprising a plurality of flavorants in a suitable excipient/carrier such that the composition possesses the odorant/flavor profile of cannabis while being substantially free of cannabinoids. In some embodiments the flavorant profile is designed to match a specific strain of cannabis and/or extracts derived therefrom.

Another embodiment is a method for providing negative control compositions for use in placebo controlled trials comprising the steps of formulating a composition having the odorant and flavor profile of a cannabis based therapeutic being tested in the same dosage form as the cannabis based therapeutic being tested.

Another embodiment is a method for testing the hypothesis that one or more whole plant extracts are responsible for a particular biological effect or effects comprising: selecting a whole plant extract to be tested for a particular biological effect or effects, creating a negative control formulation that has a substantially similar odor/flavor profile of the whole plant extract while being substantially free of cannabinoids, and running comparative tests between the whole plant extract and the negative control formulation.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context

The cannabinoids are a class of molecules primarily obtained through the extraction of cannabis plant material. The various cannabinoids include tetrahydrocannabinol (THC), cannabidiol (CBD), tetrahydrocannabinolic acid (THCA), Cannabidiolic acid (CBDA), cannabinol (CBN), cannabigerol (CBG), cannabichromene (CBC), tetrahydrocannabivarin (THCV), and cannabidivarin (CBDV) as well as others. Various cannabinoids, used alone or in combination have shown a variety of significant biological effects including but not limited to pain relief, anti cancer, anti inflammatory, anti emetic, anti convulsant, and several others. The pharmacological utility of whole plant cannabis extracts is receiving increased attention lately as evidence of ‘entourage’ effects has developed.

According to a first embodiment, the present invention provides a composition comprising a plurality of flavorants in a suitable excipient/carrier such that the composition possesses the odorant/flavor profile of cannabis and is therefore suitable for use as a negative control in placebo controlled studies of cannabis and cannabis based extracts. In some embodiments the flavorant profile is designed to match a specific strain of cannabis and/or extracts derived therefrom. The most relevant ingredients to achieve this end are the monoterpenes, diterpenes, carotenoids, phytols, tetraterpenes, sesquiterpenes, sterols, triterpenes, and flavonoids. The terpenes and terpenoids found in cannabis generally include over 120 compounds known to those having skill in the art, some of the more common include, but are not limited to: pinene, humulene, b-caryophyllene, isopulegol, guaiol, nerylacetate, neomenthylacetate, limonene, menthone, dihydrojasmone, terpinolene, menthol, phellandrene, terpinene, geranylacetate, ocimene, myrcene, 1,4-cineole, 3-carene, linalool, menthofuran, perillyalcohol, pinane, and substantial others. These various flavorants may be added to the composition of the present invention in varying ratios and amounts, such as, for example between 0.01-10% of the total composition by weight. In some embodiments the ratios of the various flavorants may be formulated to match the ratios of terpenes/terpenoids and other flavorants found in popular cannabis strains. In some cases, these ratios are known to those having skill in the art, and can be found, for example, at: https://shop.greenhouseseeds.nl/strains-terpenes-profiles.html. In other cases, the specific odorant/flavorant profile of the strain would need to be determined experimentally, such as, for example using gas chromatography or other methods known to those having skill in the art. For use as negative controls, the compositions are formulated to match the form factor of the experimental compound(s), extracts or material. By way of non-limiting illustration, form factors may include, oils for vaporization, tablets, capsules, gel caps, oral solutions, tinctures, topical formulations and the like.

Suitable excipients and/or carriers include but are not limited to medium chain triglycerides (MCT), propylene glycol, vegetable glycerin, and other excipients/carriers typically used in the electronic cigarette (e-cig) industry.

While the compositions described herein have been discussed primarily in the context of their utility as negative controls for clinical studies, they would also have utility outside of this space, such as for example for recreational use in the vaporization market. Because the compositions described mimic the flavor/odor profile of cannabis, users may enjoy the experience of vaporizing these compositions without the associated intoxicating effects of cannabis use.

Another embodiment is a method for providing negative control compositions for use in placebo controlled trials comprising the steps of formulating a composition having the odorant and flavor profile of a cannabis based therapeutic being tested in the same dosage form as the cannabis based therapeutic being tested. The general process for running placebo controlled trials is well known to those having skill in the art. Generally speaking, two or more groups are given test compositions and a non-bioactive negative control (placebo) respectively so that the effects of the test composition can be separated from the placebo effect. Prior to the invention of the present disclosure, it was difficult to adequately provide negative controls to the placebo arm of clinical trials of whole plant cannabis and cannabis extracts due to subjects' ability to detect the unique flavor and odor of the cannabis composition being used. Therefore, the compositions and methods of this invention enable scientists to adequately establish robust, well controlled clinical trials.

Another embodiment is a method for testing the hypothesis that one or more whole plant extracts are responsible for a particular biological effect or effects comprising: selecting a whole plant extract to be tested for a particular biological effect or effects, creating a negative control formulation that has a substantially similar odor/flavor profile of the whole plant extract, and running comparative tests between the whole plant extract and the negative control formulation.

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

What is claimed is:

1. A composition comprising a plurality of flavorants in a suitable excipient/carrier such that the composition possesses the odor and flavor profile of cannabis and/or a cannabis extract, while being substantially free from cannabinoids.

2. The composition of claim one wherein the odor/flavor profile is designed to match a particular strain of cannabis or extract therefrom.

3. The composition of claim 1 wherein the plurality of flavorants comprise one or more compounds selected from the group consisting of the monoterpenes, diterpenes, carotenoids, phytols, tetraterpenes, sesquiterpenes, sterols, triterpenes, and flavonoids.

4. A placebo comprising the composition of claim 1.

5. A method for providing negative control compositions for use in placebo controlled trials comprising the steps of formulating a composition having the odorant and flavor profile of a cannabis based therapeutic being tested in the same dosage form as the cannabis based therapeutic being tested.

6. The method of claim 5 wherein the cannabis based therapeutic is a whole plant extract.

7. A method for testing the hypothesis that one or more whole plant extracts are responsible for a particular biological effect or effects comprising:
selecting a whole plant extract to be tested for a particular biological effect or effects;
creating a negative control formulation that has a substantially similar odor/flavor profile of the whole plant extract while being substantially free of cannabinoids; and
running comparative tests between the whole plant extract and the negative control formulation.
